(19) United States
(12) Patent Application Publication
(10) Pub. No.: US 2017/0119402 A1
(43) Pub. Date: May 4, 2017

(54) SURGICAL INSTRUMENT

(71) Applicant: Norbert Heinemann, Spaichingen (DE)

(72) Inventor: Norbert Heinemann, Spaichingen (DE)

(21) Appl. No.: 15/204,163

(22) Filed: Jul. 7, 2016

(30) Foreign Application Priority Data
Oct. 30, 2015 (DE) 15 192 411

(51) Int. Cl. A61B 17/16; A61B 17/32

(52) U.S. Cl. CPC A61B 17/1608; A61B 17/320016; A61B 2017/00438

(57) ABSTRACT

A surgical instrument, in particular an arthroscopy punch, with a jaw (22), includes a stationary jaw part (3) and a movable jaw part (10) which, by pivoting of a pivotable grip part (6), is displaceable relative to the stationary jaw part (3), between a closed position and an open position, in a slotted guide (26) along a curved guide path.

### BACKGROUND OF THE INVENTION

The invention relates to a surgical instrument, in particular an arthroscopy punch, with a jaw, comprising a stationary jaw part and a movable jaw part which, by pivoting of a pivotable grip part, is displaceable relative to the stationary jaw part, between a closed position and an open position, in a slotted guide along a curved guide path.

Arthroscopy punches are used to remove cartilaginous joint parts, or very hard muscles or muscle strands such as the meniscus muscle. Therefore, the forces acting on the surgical instrument, and in particular on the bearing of the movable jaw part, are considerable. In order to counter this problem, U.S. Pat. No. 4,712,545 proposed a surgical instrument in which the movable jaw part is displaceable relative to the stationary jaw part in a slotted guide along a curved guide path, such that the acting forces are not just supported at individual points on a hinge axis but instead across a large surface area on a side wall of a slotted-guide groove. In this way, the considerable forces that customarily arise when using arthroscopy punches can be taken up without risking damage to the bearing of the movable jaw part, particularly also in smaller designs of arthroscopy punches in which a comparatively small amount of material is available for taking up the acting forces. The surgical instrument known from the abovementioned US specification is designed as a modified tubular shaft instrument, with a semi-open, stationary tube part and a push rod received in the tube opening, which push rod interacts with the movable jaw part via a further slotted guide. Although this ensures a stable bearing of the pull rod in the movable jaw part, the instrument cannot be dismantled for cleaning purposes. To be able to dismantle the instrument, a toolmaker with a suitable tool is needed, which means that dismantling is not feasible in practice. In addition, given the absence of guiding in translation on the tube part, the pull rod has to be configured with a relatively large diameter, which is disadvantageous especially if the instrument is for delicate work. It has also been found to be a disadvantage that, during the opening and closing movement of the jaw part, the distance between the pull rod and the upwardly open tube changes as a result of the design features. Here, there is a danger of material becoming jammed, which can adversely affect the function of the surgical instrument during the operation.

Sliding shaft instruments with a sliding part pivotable about a rotation axis are known in principle, in which a movable jaw part is pivotable about a stationary pin. The force uptake of the hinge pin for the pivotable bearing of the movable jaw part on the shaft is limited. Sliding shaft instruments of this kind are described, for example, in DE 2006 043 970 A1, DE 2 992 458 U1 or U.S. Pat. No. 5,961,531 A. In the known sliding shaft instruments, the sliding shaft, after transfer from a working position to a cleaning position, is pivotable about a rotation axis extending in a transverse direction of the instrument. A sliding shaft part permitting improved cleaning was described by the applicant in EP 2 213 254 B1, where the sliding part is rotatable about a vertical rotation axis. However, there is also the disadvantage here that considerable forces can only be transferred between sliding part and movable jaw part in a limited manner.

### SUMMARY OF THE INVENTION

The object of the invention is therefore to make available an improved surgical instrument, in particular an arthroscopy punch, which can be more easily cleaned despite the movable jaw part being mounted on a slotted guide for transferring considerable forces.

With a surgical instrument, in particular an arthroscopy punch, having the features disclosed herein, i.e. in a surgical instrument of the type in question, this object is achieved by the fact that the surgical instrument is designed not as a tubular shaft instrument but as a sliding shaft instrument, comprising a shaft assigned to the stationary jaw, preferably formed in one piece with the latter, and a sliding part which is longitudinally displaceable in translation along the shaft, in particular equidistant perpendicularly with respect to the translatory adjustment direction, by pivoting of the grip part relative to the shaft, and which is adjustable between a working position, in which it is guided longitudinally displaceably on the shaft in a guide, in particular a guide groove, and is coupled in an articulated manner to the movable jaw part for adjusting the jaw part between the closed position and the open position in a guided displacement movement of the sliding part, and a cleaning position, in which the sliding part is uncoupled from the movable jaw part, freed from the guide (relative to the shaft) and pivotable about a rotation axis relative to the shaft and relative to the movable jaw part, and by the fact that manually activatable locking means are assigned to the sliding part, with which locking means the sliding part can be secured in its working position against inadvertent transfer to the cleaning position.

Advantageous developments of the invention are set forth in the dependent claims. All combinations of at least two of the features disclosed in the description, the claims and/or the figures are covered by the scope of the invention.

The invention is based on a whole range of measures for achieving the object of the invention. Firstly, provision is made, according to the invention, to design the surgical instrument as a sliding shaft instrument, which thus comprises a sliding part that is displaceable relative to a stationary shaft, in particular a shaft that is not tubular or not partially tubular, wherein the sliding part, during its displacement movement actuated by means of the pivotable grip part, is guided longitudinally displaceably on the shaft, in particular in such a way that the distance between sliding part and shaft, measured perpendicularly with respect to the shaft width and shaft length, remains constant in the adjustment movement, since the sliding part is caught on the shaft on account of the guide.

This distance is preferably minimal or non-existent and the shaft part slides directly on the shaft. By means of these measures, a first objective is already achieved, namely that of being able to configure the sliding part with a relatively small diameter or with a small material thickness.

Furthermore, the configuration of a sliding shaft instrument in contrast to a tubular shaft instrument affords the possibility of connecting the sliding part, in a manner according to the invention, releasably and in an articulated manner to the movable jaw part provided with the slotted guide, in such a way that the sliding part can be uncoupled from the movable jaw part without use of tools, after locking means assigned at least indirectly to the sliding part have been unlocked. The locking means are necessary in order to prevent the sliding part from being able to be accidentally or inadvertently transferred from a working position to a cleaning position in which, in contrast to a pull rod of a tubular shaft instrument, the sliding part is pivotable relative to the shaft and to the movable jaw part in order to be able to more easily clean the area between sliding part and shaft.

In the working position, by contrast, the axial translatory adjustability of the sliding part relative to the shaft is limited between two extreme positions which define the closed position and open position of the jaw, wherein the open position is preferably defined by the locking means which for this purpose, for example, limit the pivoting movement of the grip part, i.e. the pivoting angle of the grip part, and hence in turn the axial adjustment of the sliding part, as a result of which the displacement position of the movable jaw part in the slotted guide is then also ultimately limited. It is therefore essential that the sliding shaft instrument is designed to allow sliding part and movable jaw part to be uncoupled without the use of tools, i.e. that a corresponding operative connection can be opened without using tools, with the result that the sliding shaft can be pivoted relative to the movable jaw part and to the shaft about a rotation axis.

In particular with regard to a reduced number of parts and to increased robustness and stability of the surgical instrument, it is particularly expedient to design the stationary jaw part as a component part of the shaft, i.e. formed in one piece with the latter, although, alternatively, it is in principle also conceivable to design the stationary jaw part as a separate component part and to rigidly connect it, in particular permanently, to the shaft.

Overall, therefore, the invention makes available a surgical instrument in which, for the first time, on account of the slotted-guide bearing of the movable jaw part relative to the stationary jaw part, in particular directly on the shaft, considerable punching forces can be transmitted (across a large surface area), and yet, by virtue of the special design of the surgical instrument as a sliding shaft instrument having a sliding part which can be coupled releasably to the movable jaw part without using tools and which in its displacement movement relative to the shaft is guided in a guide, in particular a guide groove in the shaft, it is possible to achieve small material thicknesses and, moreover, to ensure good cleaning, on account of the pivotable arrangement of the sliding part about a rotation axis, in particular after the operative connection between sliding part and movable jaw part has been released without using tools.

As has already been indicated at the outset, the sliding part, in its working position, is caught on the shaft or a guide assigned thereto, in particular a guide groove provided in the shaft, in particular in such a way that the distance (height distance), measured perpendicularly with respect to the longitudinal extent of the shaft and perpendicularly with respect to the width extent of the shaft and, as will be explained below, preferably parallel to the rotation axis, of the sliding part from the shaft is the same in the closed position and the open position of the movable jaw part and during the adjustment movement between these positions. To put it another way, the aforementioned distance from the shaft does not change, in particular along the entire longitudinal extent of the sliding part, in an adjustment of the movable jaw part while the sliding part is located in its working position and the locking means are locked. Alternatively, no above-described distance is present during the entire adjustment movement, and the sliding part slides on the shaft.

As has been mentioned, the surgical instrument according to the invention is designed, in a preferred embodiment, as a punch, in particular as an arthroscopy punch, although the invention is not expressly limited to this. Thus, the jaw or working end can, for example, also be designed as scissors with a movable scissor part and a stationary scissor part, or as forceps with a movable forceps part and a stationary forceps part.

It is particularly expedient if the coupling/operative connection between sliding part and movable jaw part is such that a coupling and/or uncoupling is possible with parallelized sliding part and shaft (after suitable unlocking of the locking means) by a simple pivoting movement of the movable grip part and a resulting translatory adjustment movement of the sliding part relative to the movable jaw part, in particular in such a way that at least one pin-shaped extension of the sliding part is, for uncoupling purposes, moved or movable out of at least one corresponding hinge recess of the movable sliding part and, for coupling purposes, is moved or movable back into this hinge recess. For this purpose, the operative connection between sliding part and movable jaw part is preferably not designed as a slotted guide but instead as a deliberately simple hinge connection, with which the movable jaw part is rotatable about a pivot axis extending through the pin extension of the sliding part and defined by same, such that displacement of the sliding part along a slotted guide is avoided for the purposes of simple coupling and uncoupling.

As regards the specific design of the slotted guide, it is preferable if the latter is designed in such a way that the movable jaw part, during its displacement movement along the curved guide path, describes a partial circle trajectory about an imaginary pivot axis, which preferably extends at a distance from the sliding shaft part, i.e. preferably does not intersect this.

It is particularly expedient if the slotted guide comprises at least one curved, in particular partially circular, slotted-guide groove, which defines the guide path and is arranged in particular on the shaft and/or in the stationary jaw part and in which an in particular curved engagement portion, preferably curved with the same radius of curvature as the slotted-guide groove and preferably provided on the movable jaw part, is displaceable along the longitudinal extent of the slotted-guide groove in the movement of the movable jaw part between the closed position and the open position. The slotted-guide track preferably extends at a radial distance from and concentric to the imaginary pivot axis.

The slotted guide preferably overall comprises two pairs of slotted guides spaced apart in the direction of the width or transverse extent of the surgical instrument, each one comprising a slotted-guide groove and, guided displaceably therein, an engagement portion which preferably bears on a groove wall, not just along a contact line extending in the transverse direction of the surgical instrument, but instead across a large surface area.

As has already been indicated, it is preferable if sliding part and movable jaw part are connected, in the working position of the sliding part, not via a slotted guide but instead via an in particular traditional hinge connection in which at least one pin extension or pin of the sliding part, extending in the direction of the width/transverse extent of the surgical instrument, engages in an open hinge recess of the shaft part, so as to be easily uncoupled therefrom, in particular by a translatory movement of the sliding part. In order to optimize the articulated connection with regard to the maximum transmissible forces, in particular exclusively in such a way that forces of over 50.96 kpond (500 N), preferably over 61.18 kpond (600 N), more preferably over 71.38 kpond (700 N) can be transmitted between sliding part and movable jaw part, it is particularly expedient if the sliding part does not bear with just a single hinge pin on the movable jaw part, but instead if two (separate) pin extensions pointing in mutually opposite directions and configured in a single line with the sliding part or secured thereon are provided on the sliding part, and each pin extension is assigned a hinge recess the movable jaw part. In this way, lever paths can be shortened and a uniform bearing or force transmission can be ensured.

It is particularly expedient if, in the working position, the sliding part engages axially, i.e. in the direction of the longitudinal extent of the shaft, in a cutout (of the movable jaw part, that is to say into the movable jaw part) which is open rearwardly in the proximal direction, wherein this cutout is delimited by two in particular parallel wall portions spaced apart in the direction of width of the instrument, which wall portions preferably each have, in particular in an upper area, a hinge recess for receiving an aforementioned extension of the sliding part.

Overall, it has proven advantageous if, in the open position, the movable jaw part, along with the stationary jaw part, spans an opening angle, in particular of less than 90°, which is limited by the locking means, in particular indirectly by direct interaction of the locking means with the pivotable grip part, and that, in particular after the locking means have been unlocked, the movable jaw part can be uncoupled from the sliding shaft by being adjusted to an unlocking (angle) position in which the movable jaw part and the stationary jaw part span an unlocking angle, in particular of greater than 90°, which is greater than the opening angle. To put it another way, by adjustment of the sliding part after it has been unlocked, the jaw can preferably be opened farther than in the working position of the sliding part, in order thereby to permit an in particular translatory outward movement of the sliding part, in particular in order to pivot the sliding part thereafter to its cleaning position.

However, particularly if the rotation axis of the sliding part is arranged in the direction of width of the sliding part, it is also conceivable that the sliding part can already be uncoupled from the movable jaw part in the open position or, alternatively, in a then optionally provided unlocking position by pivoting about the rotation axis.

According to the invention, the sliding part, in its working position, is caught on the shaft in a guide. As has likewise already been mentioned, this guide preferably comprises a guide groove. The latter can in principle be provided in the sliding part and a guide groove engagement portion provided on the shaft, wherein a reverse arrangement is preferable on account of the material thickness ratios. At any rate, it is expedient to form this guide groove as an undercut guide groove. In known sliding shaft instruments, the guide groove is for this purpose designed as a T-shaped groove. In a development of the invention, provision is now made that the guide groove is not designed as a T-shaped groove, at least not axially continuous, and has at least in one portion an undercut space which is formed adjacent to the guide groove opening and which does not have a rectangular cross section, but instead its extent in the direction of width of the instrument, in an area located closer to the guide opening, in particular a portion adjacent to the guide opening, is greater than in a portion spaced farther apart from the guide opening along the height extent of the surgical instrument, so as to be able to leave as much material as possible on the component providing the guide groove, in particular on the shaft, so as to be able to take up substantial forces. As regards the transition between the wider portion and the narrower portion of the undercut space, there are various possibilities; as regards simple production, it is preferable if the side walls are beveled for this purpose, in such a way that a trapezoidal cross-sectional contour of the undercut space results.

In a development of the invention, the choice of material can also be optimized in order to ensure an embodiment that is robust over the long term. Thus, in a particularly preferred embodiment, the movable jaw part is made from a different steel alloy than the stationary part and the shaft, in the sense that the steel alloy of the movable jaw part is made from a tougher and therefore less brittle steel material than the shaft and/or the stationary jaw part.

This can be achieved by a higher Rockwell hardness, in particular if the steel material of the stationary jaw part and of the shaft has a Rockwell hardness at least 4 HRC higher than the steel material of the steel alloy of the movable jaw part and/or a higher carbon fraction, in particular a carbon fraction at least 0.2% higher.

Overall, it is advantageous if the Rockwell hardness of the movable jaw part is chosen from a value range of between 45 HRC and 47 HRC and/or the Rockwell hardness of the steel alloy of the shaft and/or of the stationary part is chosen from a value range of between 55 HRC and 57 HRC.

Overall, it is advantageous if the sliding part is caught on the shaft by means of the guide in such a way that, after the unlocking means have been unlocked, the sliding part is not pivotable directly but instead is first adjustable still in translation to a certain extent guided away from the jaw in the direction of the proximal end of the surgical instrument. To put it another way, it is expedient if, after the locking means have been unlocked, the sliding part is adjustable in translation farther away from the jaw, in particular in such a way that the sliding part, before coming free from the guide in order to be able to be pivoted relative to the shaft and to the jaw, remains caught to a certain extent inside the guide or is adjustable in translation away from the jaw while caught inside the guide, in particular along a distance of at least 1 mm, preferably between 1 mm and 5 mm, particularly preferably between 2 mm and 3 mm. Such an embodiment is particularly advantageous when, as will be explained below, the rotation axis about which the sliding part is pivotable in the cleaning position is oriented perpendicularly with respect to the pivot axis of the grip part and perpendicularly with respect to the longitudinal axis of the shaft.

Particularly in an embodiment in which the rotation axis is oriented parallel to the pivot axis of the grip part, it is conceivable to dispense with such a caught translatory adjustment after unlocking of the locking means, and in such an embodiment it is also advantageous to provide a further translatory adjustment movement, caught inside the guide, of the sliding part before the sliding part comes free from the guide, in order then to be able to be pivoted relative to the shaft and to the movable jaw part.

As has been mentioned, it is particularly expedient if the sliding part, in its cleaning position, is arranged rotatably about a rotation axis oriented in the height direction of the instrument, i.e. perpendicularly with respect to the longitudinal extent and perpendicularly with respect to the width and thus perpendicularly with respect to the orientation of the pivot axis of the grip part, it being still more preferable if the sliding part is secured captively on the shaft both in the working position and in the cleaning position and during the pivoting movement relative to the shaft.

### BRIEF DESCRIPTION OF THE DRAWINGS

Further advantages, features and details of the invention will become clear from the following description of preferred illustrative embodiments and from the drawings, in which:

FIG. 1 shows a partially sectioned side view of a surgical instrument designed as an arthroscopy punch in the form of a sliding shaft instrument, wherein the sliding part is located in a working position in which the sliding part is guided longitudinally displaceably with respect to the shaft,

FIG. 2 shows a plan view of the surgical instrument according to FIG. 1,

FIG. 3 shows a plan view of the surgical instrument with the sliding part located in the cleaning position, which sliding part is additionally pivoted relative to the shaft and to the jaw,

FIG. 4 shows an enlarged sectional view of the distal instrument end with the jaw opened,

FIG. 5 shows a longitudinal sectional view of the movable jaw part on its own,

FIG. 6 shows a plan view of the movable jaw part according to FIG. 5,

FIG. 7 shows a cross-sectional view of a guide groove for guiding the sliding part on the shaft.

In the figures, elements that are identical and elements that have an identical function are indicated by the same reference signs.

### DETAILED DESCRIPTION

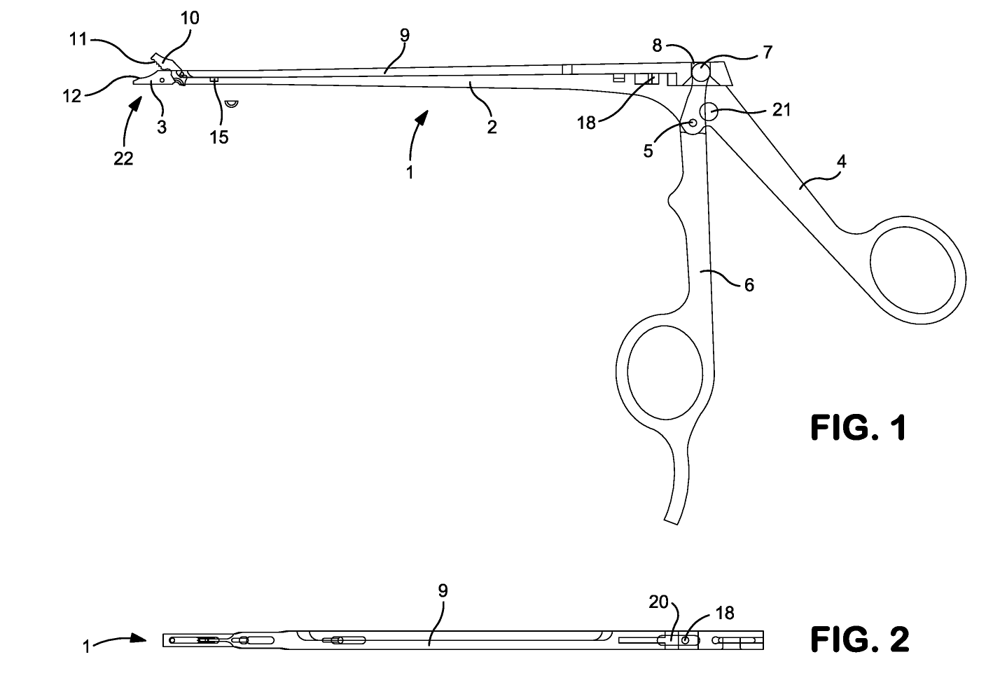

In FIGS. 1 to 3, a preferred illustrative embodiment of a surgical instrument 1 according to the invention, designed as an arthroscopy punch, is shown in its entirety. The surgical instrument 1 comprises an elongate, stationary shaft 2 with a distal (front) working end, which is partially formed by a stationary jaw part 3 formed in one piece with the shaft 2. A rear grip part 4 (grip branch) is also formed in one piece with the shaft 2.

A distal (front) pivotable grip part 6 is arranged to be pivotable, relative to the stationary rear (proximal) grip part 4, about a pivot axis 5 extending transversely with respect to the longitudinal extent of the shaft 2.

The pivotable (front) grip part 6 has an upper end 7 which has the approximate shape of a spherical head and which engages in a laterally open cutout 8 of a sliding part 9 arranged on the shaft 2. In this way, the sliding part 9 can be driven longitudinally displaceably along the longitudinal extent of the shaft 2 by means of the front pivotable grip part 6, by pivoting of the latter about the pivot axis 5 relative to the shaft 2.

The sliding part 9 is releasably connected in an articulated manner to a pivotable jaw part 10, as long as the sliding part 9 is located in a working position, which will be explained below. The pivotable jaw part 10 and the stationary jaw part 3 cooperate in the manner of a punch, by displacement of the shaft 9, and together form the distal working end (jaw). For this purpose, the jaw parts 3, 10 have cutting surfaces/punching surfaces 11, 12 facing toward each other.

The sliding part 9 is shown in its working position in FIGS. 1 and 2. In the working position, the sliding part 12 is guided longitudinally displaceably on the shaft 2. As a guide 13 in the area of the distal end area of the shaft 2, a guide groove 14 is provided in which a lower guide groove engagement portion 15 in the area of the distal end area of the sliding part 9 engages. The guide 13 is configured such that the sliding part 9, in its working position, is caught in the guide 13, more precisely in the guide groove 14, over the entire longitudinal displacement path along the shaft, i.e. including in a distal end position and in a proximal end position, which positions define the pivoting angle or opening angle of the jaw parts 3, 10 relative to each other.

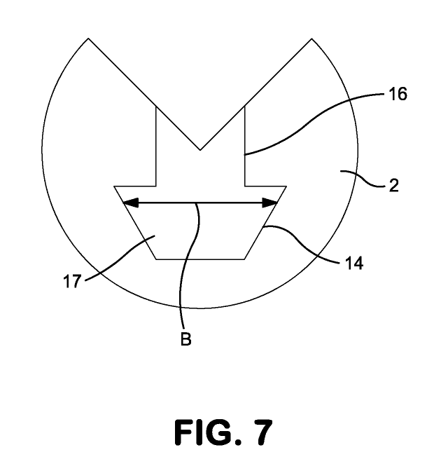

For a preferred embodiment of the guide groove 14 and of the guide 13, reference is made to FIG. 7.

A cross-sectional view of the shaft 2 can be seen here. The guide groove 14, open toward the top and designed as an undercut groove, is located in this shaft 2.

The guide groove 14 is not designed traditionally as a T-shaped groove, but instead an undercut space 17 arranged underneath a guide groove opening 16 has a trapezoidal cross-sectional contour. For this purpose, the undercut space 17 is wider in the direction of width B of the shaft 2 in an area, here an upper area, adjoining the guide groove opening 16 than it is in a more remote (lower) area, i.e. in the area of the bottom of the groove. The side walls of the groove are beveled, resulting in the trapezoidal shape. For this purpose, a comparatively large amount of material (steel alloy) is present in the area that adjoins the undercut space laterally, or in the direction of width B, such that, overall, substantial guiding forces can be taken up without risking damage to the shaft 2.

To make the surgical instrument easier to clean, a rotation axis 18 is provided for the sliding part 9. However, a pivoting movement about this rotation axis 18 is not possible in the working position shown in FIGS. 1 and 2, in which position the sliding part 2 is caught in the guide 13. As will be seen from the figures, the rotation axis 18, embodied for example by a head screw 19 or alternatively a head rivet, passes through an elongate hole 20 in the sliding part 9, such that the sliding part 9 is longitudinally displaceable relative to the rotation axis 18. The head screw 18 is fixed in position relative to the shaft 2.

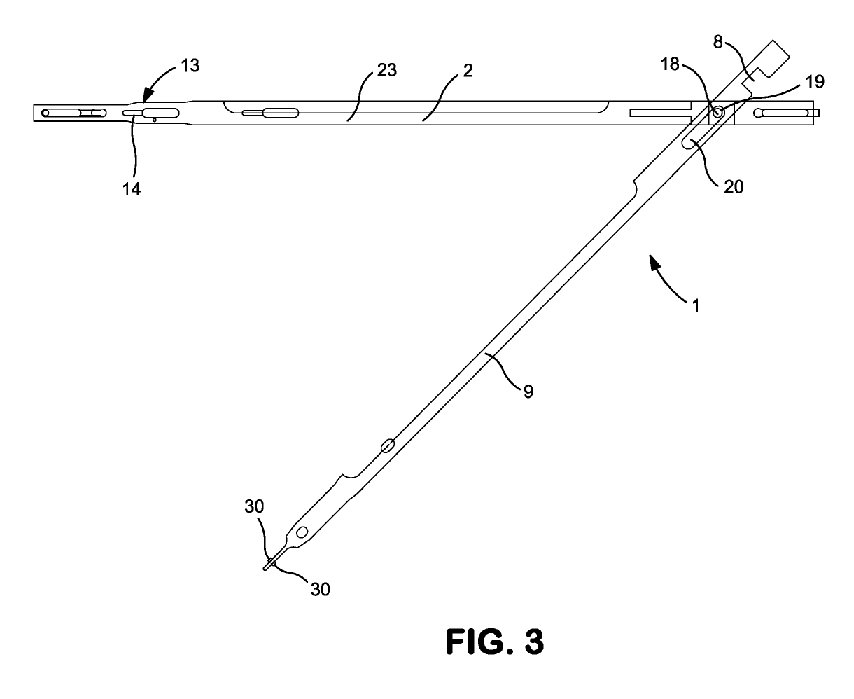

The sliding part 9 is assigned locking means 21, which secure the sliding part 9 in the working position shown in FIG. 1, in which position it is adjustable exclusively in translation relative to the shaft 2, specifically by pivoting of the movable grip part 6 in order to open and close the jaw 22, which is formed by the jaw parts 3, 10. For this purpose, the locking means 21 limit the maximum pivoting angle of the movable grip part. By actuation or unlocking of the locking means 21, the maximum pivoting angle is increased, as a result of which the sliding part 9 comes free from the guide 13 or is adjustable to a cleaning position (cf. FIG. 3) in which it is pivotable about the rotation axis 18 which is oriented perpendicularly with respect to the pivot axis 5 and perpendicularly with respect to the longitudinal extent of the shaft 2.

FIG. 3 shows the surgical instrument in its cleaning position with the sliding part 9 pivoted. It will be seen that an upper face 23 of the shaft, otherwise covered by the sliding part 9, can be optimally cleaned in this pivoted state. The same applies to a lower face (not visible in FIG. 3) of the sliding part directed toward the upper face 23 of the shaft.

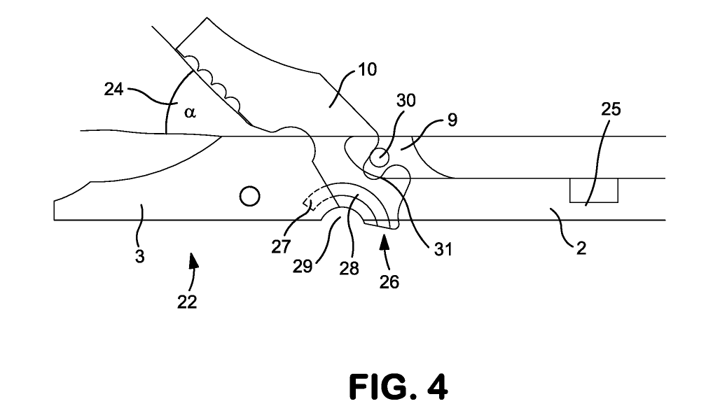

In order to permit a pivoting movement of the sliding part 9, the latter, as will be explained below, has to be uncoupled from the movable jaw part 10 without using tools. At the same time, the cutout 8 interacting with the pivotable grip part is designed open toward the sides, as can be seen from FIG. 3, so as to be able to be pivoted laterally away from the active area of the pivotable grip part 6. As will be seen from FIG. 4, a laterally open window 25 is also provided in the shaft 2 in the proximal direction from the guide groove, through which window 25 the trapezoidally contoured guide groove engagement portion of the sliding part 9 can be pivoted laterally outward.

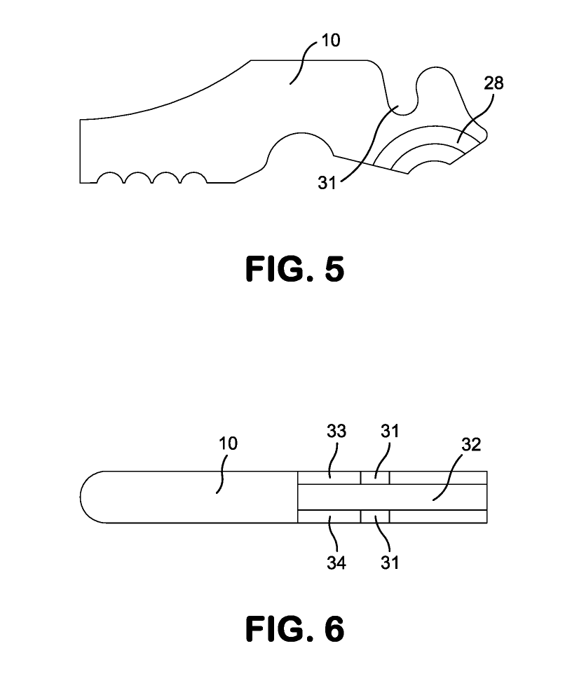

The releasable coupling between sliding part 9 and movable (pivotable) jaw part 10 and the bearing of the jaw part 10 on the shaft 2 are explained in detail below with reference to FIGS. 4 to 5.

It is also essential that the movable jaw part 10 is guided on the shaft 2 via a slotted guide 26. The slotted guide 26 comprises two slotted-guide grooves 27 which are curved in the shape of part of a circle in the shaft, along the width of the shaft 2, and within which, by adjustment of the sliding part 9, the movable jaw part 10 is displaceable by means of an engagement portion formed in one piece with the movable jaw part 10. The engagement portion can thus be displaced inside the partially circular guide groove 27 and is thereby pivotable relative to the stationary jaw part 3 about an imaginary pivot axis 29. Specifically, two curved engagement portions 28 having a shape congruent to the slotted-guide groove 27 and protruding in mutually opposite width directions are provided on the movable jaw part 10 and engage in the open slotted-guide groove 27 of the shaft 2, or the slotted-guide groove 27 directed respectively to the associated engagement portion 28 of the shaft 2, thus ensuring that force is taken up across a large surface area. In a pivoting movement of the movable jaw part, the engagement portion thereof migrates along the slotted-guide groove and, in this movement, also includes a translatory movement component (vector).

In the working position, as has been mentioned, the sliding part 9 is coupled or operatively connected to the movable jaw part 10, specifically via two pin extensions 30 which protrude laterally, or in the direction of width of the instrument, and which are each assigned a hinge recess 31 for formation of pivot hinges. For this purpose, the pivotable jaw part 10 has a distally open cutout 32 into which the distal end area of the sliding part 9 engages in the working position of the latter, in such a way that the pin extensions 30 is received in a hinge recess 31 in each case. The hinge recesses 31 are received in parallel wall portions 33, 34 of the movable jaw part 10, which wall portions 33, 34 laterally delimit the cutout 32.

FIG. 4 shows a maximum opening angle α of the jaw 22 with the sliding part 9 located in the working position. That is to say, a further translatory adjustment movement of the sliding part 9 for further opening of the jaw 22 is prevented by the locking means. After the locking means have been unlocked, the sliding part 9 can be moved farther to the right in the drawing plane, i.e. in the proximal direction, as a result of which an unlocking angle of the jaw parts 3, 10 relative to each other is obtained that is greater than the maximum opening angle 24 (a) in the working position. In this way, the pin extensions 30 can then be automatically moved out of the respectively assigned hinge recess 31 in the proximal direction, as a result of which the sliding part comes free.

According to a preferred embodiment realized here, the sliding part 9 is not pivotable directly after the locking means have been unlocked, but instead it here has to be displaced farther in the proximal direction, on the one hand in order to come free from the hinge recesses 31 and, which is important, also to come free from the guide groove 14, which is achieved when the merely by way of example trapezoidal guide groove engagement portion of the sliding part 2 is laterally flush with the window 25 in the shaft 2.

1. Surgical instrument, in particular an arthroscopy punch, with a jaw (22), comprising a stationary jaw part (3) and a movable jaw part (10) which, by pivoting of a pivotable grip part (6), is displaceable relative to the stationary jaw part (3), between a closed position and an open position, in a slotted guide (26) along a curved guide path, wherein the surgical instrument (1) is designed as a sliding shaft instrument, comprising a shaft (2) assigned to the stationary jaw part (3) and a sliding part (9) which is longitudinally displaceable in translation along the shaft (2), by pivoting of the grip part (4, 6) relative to the shaft (2), and which is adjustable between a working position, in which it is guided longitudinally displaceably on the shaft (2) in a guide (13) and is coupled in an articulated manner to the movable jaw part (10) for adjusting the jaw part (3, 10) between the closed position and the open position in a guided displacement movement of the sliding part (9), and a cleaning position, in which the sliding part (9) is uncoupled from the movable jaw part (10), freed from the guide (13) and pivotable about a rotation axis (18) relative to the shaft (2) and relative to the movable jaw part (3), and in that manually activatable locking means (21) are assigned to the sliding part (9), with which locking means (21) the sliding part (9) can be secured in its working position against inadvertent transfer to the cleaning position.

2. Surgical instrument according to claim 1, wherein a distance, measured perpendicularly with respect to the longitudinal extent of the shaft (2) and parallel to the rotation axis (18), of the sliding part (9) from the shaft (2) is the same in the closed position and the open position of the movable jaw part (10) and during the adjustment movement between these positions, or wherein the sliding part (9), in the closed position and the open position of the movable jaw part (10) and during the adjustment movement, bears on a shaft face directed toward the shaft part (9).

3. Surgical instrument according to claim 1, wherein, with sliding part (9) and shaft (2) oriented parallel to each other, the sliding part (9) can be uncoupled from the movable jaw part (10), after the locking means (21) have been unlocked, by adjustment of the sliding part (9) away from the jaw (22) by means of the pivotable grip part (6) and/or can be coupled to the movable jaw part (10) in an articulated manner by adjustment of the sliding part (9) in the direction of the jaw (22) by means of the pivotable grip part (6).

4. Surgical instrument according to claim 1, wherein the slotted guide (26) is designed in such a way that the movable jaw part (10), during its displacement movement along a partial circle trajectory, is adjustable about an imaginary pivot axis (29).

5. Surgical instrument according to claim 1, wherein the slotted guide (26) comprises at least one curved slotted-guide groove (27), which defines the guide path and is provided in particular on the shaft and/or stationary jaw part and in which an in particular curved and movable engagement portion (28) on the jaw part is displaceable along the longitudinal extent of the slotted-guide groove in the movement of the movable jaw part (10) between the closed position and the open position.

6. Surgical instrument according to claim 1, wherein the sliding part (9) has two lateral pin extensions (30) pointing in mutually opposite directions and oriented perpendicularly with respect to the longitudinal axis of the shaft (2), which pin extensions (30) engage in each case in an associated open hinge recess (31) of the movable jaw part (10).

7. Surgical instrument according to claim 1, wherein the sliding part (9), in its working position along the longitudinal extent of the shaft (2), protrudes into a cutout (32) which is open toward the proximal end of the instrument and which is delimited by two wall portions (33, 34) spaced apart along a transverse direction extending perpendicularly with respect to the longitudinal extent of the shaft (2).

8. Surgical instrument according to claim 1, wherein in the open position, the movable jaw part (10), along with the stationary jaw part (3), spans an opening angle (α) of less than 90°, which is limited by the locking means (21), and wherein after the locking means (21) have been unlocked, the movable jaw part (10) can be uncoupled from the sliding shaft by being adjusted to an unlocking position in which the movable jaw part (10) and the stationary jaw part (3) span an unlocking angle of greater than 90°, which is greater than the opening angle.

9. Surgical instrument according to claim 1, wherein the guide (13) for the longitudinally displaceable guiding of the sliding part (9) on the shaft (2) has an undercut guide groove (14) in the shaft (2) with a guide groove opening (16) extending in the direction of the longitudinal extent of the shaft, and wherein, adjacent to the guide groove opening (16), an undercut space (17) is formed in which, in the working position, a guide groove engagement portion (15) of the sliding part (9) is guided longitudinally displaceably, and of which the width of the undercut space measured perpendicularly with respect to the longitudinal extent of the shaft and perpendicularly with respect to the depth of the guide groove, in the direction of the guide groove depth extent, is smaller area spaced farther apart from the guide groove opening (16) than in an area located closer to the guide groove opening (16).

10. Surgical instrument according to claim 9, wherein a cross-sectional contour of the undercut space (17) and/or of the guide groove engagement portion (15) is trapezoidal.

11. Surgical instrument according to claim 1, wherein the movable jaw part (10) and the shaft (2) and also the stationary jaw part (3) are each made from a steel alloy, wherein the steel alloy of the shaft (2) and of the stationary jaw part (3) has a higher carbon fraction and/or an at least 5 HRC higher Rockwell hardness than the steel alloy of the movable jaw part (10).

12. Surgical instrument according to claim 1, wherein, after the locking means (21) have been unlocked, the sliding part (9) is adjustable in translation farther away from the jaw (22) in such a way that the sliding part (9), before coming free from the guide (13) in order to be able to be pivoted relative to the shaft (2) and to the jaw (22), is adjustable in translation away from the jaw (22) to a certain extent while caught inside the guide (13) along a distance of at least 1 mm, preferably between 1 mm and 5 mm.

13. Surgical instrument according to claim 1, wherein the rotation axis (18), about which the sliding part (9) is pivotable in the cleaning position, is oriented perpendicularly with respect to the pivot axis (5) of the grip part (4, 5) and also perpendicularly with respect to the longitudinal extent of the shaft.

14. Surgical instrument according to claim 1, wherein the sliding shaft is secured captively on the shaft (2) both in the working position and in the cleaning position, during the transfer from the working position to the cleaning position, and also during the pivoting movement.

15. Surgical instrument according to claim 11, wherein the steel alloy of the shaft (2) has an at least 0.2% higher carbon fraction than the steel alloy of the movable jaw part (10).

16. Surgical instrument according to claim 11, wherein the steel alloy of the shaft (2) and of the stationary jaw part (3) is a 40.34 steel alloy, and wherein the steel alloy of the movavble jaw part (10) is a 40.21 steel alloy.

17. Surgical instrument according to claim 12, wherein the distance is between 1 mm and 5 mm.

18. Surgical instrument according to claim 12, wherein the distance is between 2 mm and 3 mm.
